(19) United States
(12) Patent Application Publication
(10) Pub. No.: US 2023/0301507 A1
(43) Pub. Date: Sep. 28, 2023

(54) CONTROL SYSTEM FOR AN IMAGING SYSTEM, IMAGING SYSTEM AND METHOD FOR IMAGING

(71) Applicant: LEICA MICROSYSTEMS INC., Buffalo Grove, IL (US)

(72) Inventors: Eric LYNCH, Apex, NC (US); John PERNG, St. Gallen (CH)

(21) Appl. No.: 18/019,522

(22) PCT Filed: Jul. 30, 2021
(86) PCT No.: PCT/EP2021/071474

§ 371 Date: Feb. 3, 2023

(30) Foreign Application Priority Data
Aug. 5, 2020 (DE) 10 2020 120 657.7

(51) Int. Cl. A61B 3/10; A61B 3/14

(52) U.S. Cl. CPC A61B 3/102; A61B 3/145

(57) ABSTRACT

The invention relates to a control system (130) for an imaging system (100) for real-time imaging of a subject (190), using optical coherence tomography and video imaging, the control system (130) being configured to: control the imaging system (100) to determine, from a video image (152) of the subject (190), a position and/or an orientation (152) of a tissue of interest (192) in the subject (190), control the imaging system (100) to perform a scan of the subject (190) by means of optical coherence tomography, wherein a position and/or an orientation (144) of the scan is determined based on the position and/or orientation (154) of the tissue of interest (192) in the video image (152), and provide an optical coherence tomography image (142) of the subject (190), including the tissue of interest (192), based on the scan, to an imaging system (100) and a corresponding method.

### CROSS-REFERENCE TO RELATED APPLICATIONS

The present application is a national phase entry under 35 USC § 371 of International Application PCT/EP2021/071474, filed Jul. 30, 2021, which claims the benefit of and priority to German Patent Application No. 10 2020 120 657.7 filed Aug. 5, 2020, the entire disclosure of which is incorporated herein by reference.

### TECHNICAL FIELD

The present disclosure essentially relates to a control system for an imaging system for real-time imaging of a subject, using optical coherence tomography (OCT) and video imaging, to an imaging system including such a control system, and to a method for imaging a subject in real-time, using OCT and video imaging.

### BACKGROUND

Optical coherence tomography (in the following also called OCT, its typical abbreviation) is an imaging technique that uses low-coherence light to capture two- and three-dimensional images from within optical scattering media (e.g., biological tissue) with high resolution. It is, inter alia, used for medical imaging. Optical coherence tomography is based on low-coherence interferometry, typically employing near-infrared light. The use of relatively long wavelength light allows it to penetrate into the scattering medium.

A medical field of particular interest for OCT is ophthalmology, a branch of medicine related to (in particular human) eyes and its disorders and related surgeries. A particular type of surgery performed on eyes is a so-called Descemet Membrane Endothelial Keratoplasty (in the following also called DMEK, its typical abbreviation), a technique in which an isolated Descemet's membrane is transplanted, i.e., in which a Descemet's membrane is replaced by another Descemet's membrane or a graft Descemet's membrane. The DMEK procedure is a ‘like for like’ replacement of the diseased part of the cornea of the eye with visual rehabilitation.

### SUMMARY

According to the disclosure, a control system, an imaging system and a method for imaging a subject with the features of the independent claims are proposed. Advantageous further developments form the subject matter of the dependent claims and of the subsequent description.

The present disclosure relates to a control system for an imaging system for real-time imaging of a subject using optical coherence tomography (OCT) and video imaging. This subject, preferably, includes or is an eye in which, e.g., a DMEK surgery shall be performed. Video imaging can be used to acquire a real-time image of the surface of the subject and/or a tissue of interest in the subject, and OCT can be used to acquire a (cross) sectional image of the subject or a tissue of interest in the subject. In particular, the OCT scan can include a so-called B-scan.

In OCT, areas of the sample or tissue that reflect back a lot of light will create greater interference than areas that do not. Any light that is outside the short coherence length will not interfere. This reflectivity profile is called an A-scan and contains information about the spatial dimensions and location of structures within the sample or tissue. A cross-sectional tomograph, called B-scan, may be achieved by laterally combining a series of these axial depth scans (A-scan).

During DMEK surgery, it is important to place the Descemet's membrane with the correct orientation and ensure the absence of interfacial fluid or air bubble between the graft and stroma. Wrong graft orientation leads to tissue rejection, infection and other complications and usually requires the surgery to be performed again. Interfacial fluid or air bubble also cause graft detachment and sub-optimal clinical outcome. Intraoperative OCT can be used in DMEK surgery to determine the Descemet's membrane graft orientation and to check the presence of interfacial fluid or air bubble after graft placement. However, a typical process, up to now, requires tedious manipulation by the surgeon or additional assistant on an OCT user interface.

It has now turned out that such problems can be overcome when, as an aspect of the disclosure, the control system is configured to control the imaging system in a special way. Firstly, the control system is configured to determine, from a video image of the subject, a position and/or an orientation of a tissue of interest in the subject, which can, in particular include or be a Descemet's membrane or a graft Descemet's membrane. The control system can also be configured to control the imaging system to acquire the video image of the subject by means of video imaging means like a camera.

In addition, the control system is configured to control the imaging system to perform a scan of the subject by means of optical coherence tomography, wherein a position and/or an orientation of the scan is determined based on the position and/or orientation of the tissue of interest in the video image. Which position and/or orientation exactly will be used for the OCT scan can be selected, e.g., upon specific requirements of the particular application (or surgery). For example, for a DMEK surgery, the OCT scan should be performed orthogonally to the orientation of the (graft) Descemet's membrane. Further, the control system is configured to provide an optical coherence tomography (OCT) image of the subject, including the tissue of interest, based on the scan. In this way, the OCT scan can be performed (automatically) at the right (and required) position and/or orientation and at the area of interest and display the information via, e.g., image overlay, and thus time is saved and workflow efficiency is improved since the surgeon is provided with the optimal OCT image automatically.

It is also to be noted that this procedure can be performed (by means of the control system, which can be configured accordingly) repeatedly, such that the position and/or orientation based on which the OCT scan is to be performed is (continuously) updated. In this way, the problem of movements (of the subject and/or tissue of interest) during, e.g., surgery can easily be overcome.

Preferably, the control system is configured to determine, from the video image, the position and/or orientation of the tissue of interest in the subject by means of feature tracking and/or artificial intelligence (or machine learning, using, e.g., a machine learning model). Feature tracking can be based, e.g., on recognizing edges in the video image that indicate the position and/or orientation of the tissue of interest. Artificial intelligence can use, e.g., artificial neural networks which receive the video image as input and provide the position and/or orientation as output. Such artificial neural networks can, for example, initially (before first use) be trained using test video images and providing information about the position and/or orientation therein.

According to a further aspect of the disclosure, the control system is configured to control the imaging system to perform a multitude of different scans of the subject, to identify undesired objects present in the subject, and to indicate the undesired objects within the video image of the subject, e.g., by means of overlay. Such undesired objects can be, e.g., air bubbles and/or interfacial fluid (fluid between, for example, the graft Descemet's membrane and the stroma of the eye) as mentioned for the example above. Due to such (automatic) detection of such objects the surgery can be made faster and with better clinical outcome.

It is to be noted that this multitude of OCT scans and the indication of undesired objects in the video image can be carried out in addition to determining the position and/or orientation and the corresponding OCT scan, and it can also be carried out without such position and/or orientation determination, as a separate or further aspect of the disclosure.

This further aspect of the disclosure relates to a control system for an imaging system for real-time imaging of a subject using optical coherence tomography and video imaging, the control system being configured to control the imaging system to perform a multitude of different scans of the subject, to identify undesired objects present in the subject, and to indicate the undesired objects within the video image of the subject.

Preferably, the control system is configured to identify the undesired objects by means of feature tracking and/or artificial intelligence (or machine learning, using, e.g., a machine learning model). Feature tracking can be based, e.g., on recognizing edges in the video image that indicate the position of the air bubbles and interfaces or the like. Artificial intelligence can use, e.g., artificial neural networks which receive the video image as input and provide the position of air bubbles and interfaces or the like as output. Such artificial neural networks can, for example, initially (before first use) be trained using test video images and providing information about the position of such undesired objects therein.

The disclosure also relates to an imaging system for real-time imaging of a subject, e.g. an eye, comprising the control system according to the disclosure and as described above, optical coherence tomography means in order to perform the OCT scan (for a more detailed description of such OCT means it is referred to the drawings and the corresponding description), and video imaging means (e.g., a video camera) in order to acquire an image (real-time image) of the subject.

Further, the imaging system is, preferably, configured for use during a surgical procedure being performed on the subject like an eye of a patient. As mentioned before, the (automatic) adaption of the OCT scan based on the current position and/or orientation of the tissue of interest used within the present disclosure allows an intraoperative use, i.e., an OCT image of the tissue material of interest like the Descemet's membrane can be visualized next to a surface image of the subject correctly and without time delay during the surgery.

The disclosure also relates to a method for imaging a subject like an eye in real-time, using optical coherence tomography (OCT) and video imaging. The method comprises the following steps: from a video image of the subject, a position and/or an orientation of a tissue of interest like a (graft) Descemet's membrane in the subject are determined, and a scan the subject is performed by means of OCT, wherein a position and/or an orientation of the scan is determined based on the position and/or orientation of the tissue of interest in the video image. Further, an OCT image of the subject is provided, the OCT image including the tissue of interest, based on the scan. The method, preferably, further (or alternatively) comprises the steps of performing a multitude of different scans of the subject, identifying undesired objects like air bubbles and/or interfacial fluids present in the subject, and indicating the undesired objects within the video image of the subject. This multitude of different scans of the subject is, in particular, equally distributed with respect to an orientation and/or covers 360°.

With respect to further preferred details and advantages of the imaging system and the method, it is also referred to the remarks for the control system above, which apply here correspondingly.

The disclosure also relates to a computer program with a program code for performing a method according to the disclosure when the computer program is run on a processor or on a control system according to the disclosure.

Further advantages and embodiments of the disclosure will become apparent from the description and the appended figures.

It should be noted that the previously mentioned features and the features to be further described in the following are usable not only in the respectively indicated combination, but also in further combinations or taken alone, without departing from the scope of the present disclosure.

### SHORT DESCRIPTION OF THE FIGURES

FIG. 1 shows a schematic overview of an imaging system according to the disclosure in a preferred embodiment.

FIG. 2 shows a video image and an OCT image for explanation of an aspect of the disclosure.

FIG. 3 shows a video image and an OCT image for explanation of a further aspect of the disclosure.

FIG. 4 shows, schematically, a flow scheme describing a method according to the disclosure in a preferred embodiment.

### DETAILED DESCRIPTION

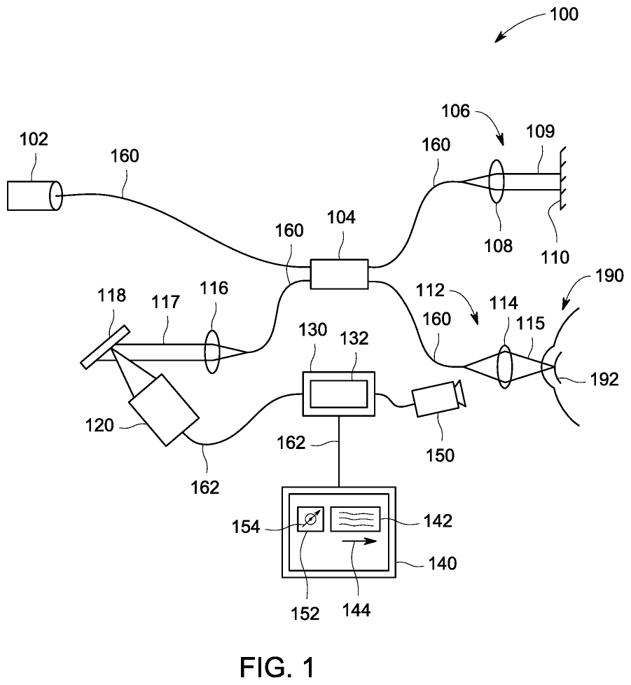

In FIG. 1, a schematic overview of an imaging system 100 according to the disclosure in a preferred embodiment is shown. The imaging system 100 comprises a light source 102 (e.g., a low coherence light source), a beam splitter 104, a reference arm 106, a sample arm 112, a diffraction grating 118, a detector 120 (e.g., a camera), a control system 130, display means 140 (e.g., a display or monitor), and video imaging means 150 (e.g., a camera). In particular components 102, 104, 106, 112 and 120 and related components can be regarded as OCT means.

Light originating from the light source 102 is guided, e.g., via fiber optic cables 160, to the beam splitter 104 and a first part of the light is transmitted through the beam splitter 104 and is then guided, via a lens 108 (which is only schematically shown and may represent also different, appropriate optics) in order to create a light beam 109 to a reference mirror 110, wherein the lens 106 and the reference mirror 110 are part of the reference arm 106.

Light reflected from the reference mirror 110 is guided back to the beam splitter 104 and is transmitted through the beam splitter 104 and is then guided, via a lens 116 (which is only schematically shown and may represent also different, appropriate optics) in order to create a light beam 117 to the diffraction grating 118.

A second part of the light, originating from the light source 102 and transmitted through the beam splitter 104 and is guided, via a lens 114 (which is only schematically shown and may represent also different, appropriate optics) in order to create a light beam 115 (for scanning) to the subject 190 to be imaged and in which tissue of interest 192 is present. The lens 114 is part of the sample arm 112.

Light reflected from the subject 190 or the tissue of interest 192 is guided back to the beam splitter 104 and is transmitted through the beam splitter 104 and is then guided, via lens 116 to the diffraction grating 118. Thus, light reflected in the reference arm 106 and light reflected in the sample arm 112 are combined by means of the beam splitter 104 and are guided, e.g., via a fiber optic cable 160, and in a combined light beam 117 to the diffraction grating 118.

Light reaching the diffraction grating 118 is diffracted and captured by the detector 120. In this way, the detector 120, which acts as a spectrometer, creates or acquires intensity scan data that are transmitted, e.g., via an electrical cable 162, to the control system 130 comprising processing means (or a processor) 132. The intensity scan data is then processed to obtain OCT image data that are transmitted, e.g., via an electrical cable 162, to the display means 140 and displayed as a OCT image 142, i.e., an image that represents the currently scanned subject 190 in real-time.

In addition, the video imaging means (or camera) 150 acquire a video image of the subject 190 (and, typically, also of the tissue of interest 192 in the subject) and provide corresponding video image data to the processing means 130 the video imaging means are connected to. The video image data are transmitted, e.g., via the electrical cable 162, to the display means 140 and displayed as a video image 152, i.e., a video image that represents the currently viewed subject 190 in real-time.

According to an aspect of the disclosure, a position and/or an orientation of the subject 190 are determined in the video image 152. By means of example, the orientation of subject 190 in the video image 152 is represented by arrow 154. Based on, e.g., orientation 154 an orientation 144 for the OCT scan is determined and, the OCT scan is performed in accordance with orientation 144 and a corresponding OCT image 142 is provided and displayed.

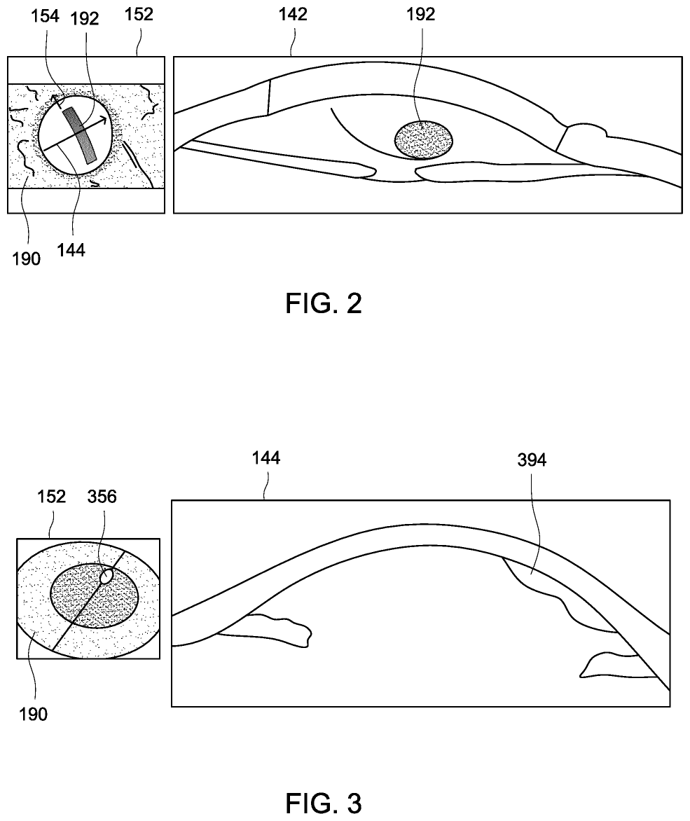

In FIG. 2, a video image 152 and an OCT image 142 corresponding to the images of FIG. 1 are shown in greater detail in order to explain an aspect of the disclosure. The video image 152 and the OCT image 142 can be presented or visualized on the display means 140 as described with respect to FIG. 1.

In video image 152, the subject 190, an eye, is shown as captured by the video imaging means mentioned before. In the subject 190, a tissue of interest 192, e.g., a graft Descemet's membrane is visible. Note that the graft Descemet's membrane has (in this top or end face view) a rectangular shape as it is rolled up and, thus, has (in 3D) a cylindrical shape. Arrow 154 indicates the orientation of the tissue 192 as also shown in FIG. 1. This orientation 154 can be determined within the present disclosure by, e.g., feature tracking (recognizing edges due to different colors and/or brightness in the image or the like), as mentioned above.

Based on the orientation 154 of the tissue, an orientation of the OCT B-scan to be performed on the subject 190 can be determined. For example, the orientation of the OCT B-scan shall be orthogonal to the orientation 154 as indicated with arrow 144 in the video image 152 (note that, in FIG. 1, orientation 144 is shown below OCT image 142).

OCT image 142 shown on the right (in FIG. 2) is based on an OCT B-scan performed according to orientation 144. The tissue 192 can be seen in the OCT image 142 in form of a rolled up flat tissue as it is typical for a graft Descemet's membrane after insertion into an eye. Due to the (automatic) determination of the orientation of the OCT scan, the OCT image 154 shows the required sectional view.

In FIG. 3, a video image 152 and an OCT image 142 basically corresponding to the images of FIG. 1 are shown in greater detail in order to explain another aspect of the disclosure. The video image 152 and the OCT image 142 can be presented or visualized on the display means 140 as described with respect to FIG. 1.

In video image 152, the subject 190, an eye, is shown as captured by the video imaging means mentioned before. Further, in video image 152, an oval spot 356 is overlaid, which spot 356 indicates the presence of, e.g., an air bubble or another undesired object in the subject 190. The position and/or geometry of this sport 356 can be determined from a multitude of OCT B-scans and, e.g., feature tracking (recognizing edges due to different brightness in the image or the like), as mentioned above. In OCT image 142 (in FIG. 3) such air bubble 394 is shown.

It is to be noted that this single OCT image 142 shows just one of the multitude of OCT B-scans used for identifying undesired objects. Also, the presentation of any of these OCT images (like OCT image 142) is not necessary for determining the position and/or geometry of the spot 356. This helps, e.g., the surgeon to check and avoid missing any interfacial fluid or other undesired objects. Note, that also further spots like spot 356 can be overlaid in video image 152, depending on the number of undesired objects in the subject 190.

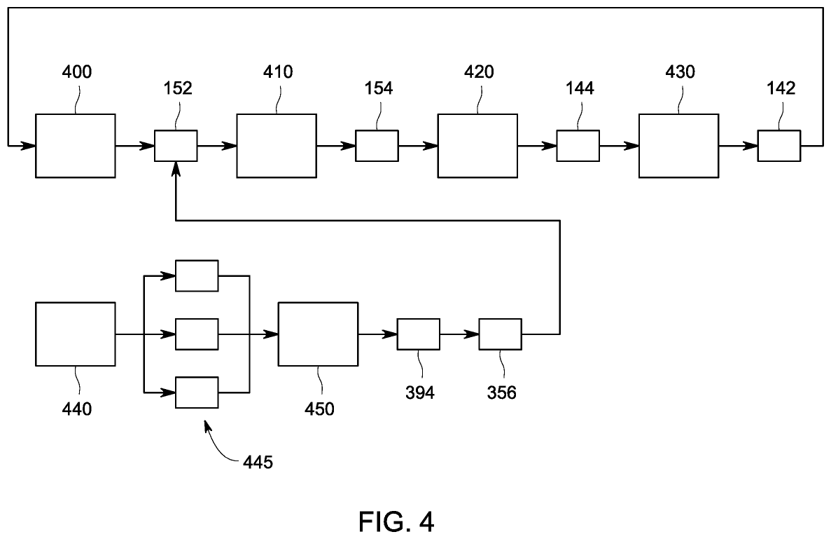

In FIG. 4, a flow scheme describing a method according to the disclosure in a preferred embodiment using, e.g., the imaging system shown in FIG. 1 is shown. In a first step 400, the subject is imaged by means of the video imaging means (or camera) as described with respect to FIG. 1, including acquiring video image data, being transmitted to the control system with its processing means. Then, a video image 152 is generated and, e.g., presented on the display means.

In a next step 410, from the video image 152, e.g., an orientation 154 of the tissue of interest in the subject is determined using, e.g., feature tracking, as described with respect to, e.g., FIG. 2. Based on this orientation 154 of the tissue of interest, in a next step 420, e.g., an orientation 144 of the OCT scan to be performed is determined. As described before, such orientation 144 can be, for example, orthogonal to the orientation 154.

In a next step 430, the subject is scanned by means of OCT based on orientation 144, as described with respect to FIG. 1, including acquiring intensity scan data, being transmitted to the control system with its processing means. Then, an OCT image 142 is generated and, e.g., presented on the display means. The result is a real-time OCT image 142 presented by the display means. These steps can be repeatedly or continuously performed such that the real-time image is repeatedly updated or is a live image of the subject showing any changes in the subject in real-time.

In parallel to steps 400 to 430 or alternatively to steps 410 to 430, in a step 440 a multitude 445 of OCT B-scans is performed on the subject. These OCT scans can cover, e.g., 360° and be equally distributed. From these B-scans, in a step 450, undesired objects 394 in the subject and their position and/or geometry are identified as described with respect to FIG. 3. From position and/or geometry of these undesired objects 394, an overlay 356 is determined which is then overlaid in the video image 152. These steps can be repeatedly or continuously performed such that the overlay in the real-time image is repeatedly updated changes of the undesired objects in real-time.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

Some embodiments relate to an imaging system comprising a control system as described in connection with one or more of the FIGS. 1 to 4. Alternatively, an imaging system may be part of or connected to a system as described in connection with one or more of the FIGS. 1 to 4. FIG. 1 shows a schematic illustration of an imaging system 100 configured to perform a method described herein. The imaging system 100 comprises an OCT system, video imaging means 150 and a computer or control system 130. The imaging system is configured to take images and is connected to the computer system 130. The computer system 130 is configured to execute at least a part of a method described herein. The computer system 130 may be configured to execute a machine learning algorithm. The computer system 130 and imaging system 100 may be separate entities but can also be integrated together in one common housing. The computer system 130 may be part of a central processing system of the imaging system 100 and/or the computer system 130 may be part of a subcomponent of the imaging system 100, such as a sensor, an actor, a camera or an illumination unit, etc. of the imaging system 100.

The computer system 130 may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system 130 may comprise any circuit or combination of circuits. In one embodiment, the computer system 130 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g. camera) or any other type of processor or processing circuit. Other types of circuits that may be included in the computer system 130 may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system 130 may include one or more storage devices, which may include one or more memory elements suitable to the particular application, such as a main memory in the form of random access memory (RAM), one or more hard drives, and/or one or more drives that handle removable media such as compact disks (CD), flash memory cards, digital video disk (DVD), and the like. The computer system 130 may also include a display device, one or more speakers, and a keyboard and/or controller, which can include a mouse, trackball, touch screen, voice-recognition device, or any other device that permits a system user to input information into and receive information from the computer system 130.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the disclosure can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the disclosure comprise a data carrier having electronically readable control signals, which are capable of cooperating with a programmable computer system, such that one of the methods described herein is performed.

Generally, embodiments of the present disclosure can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present disclosure is, therefore, a computer program having a program code for performing one of the methods described herein, when the computer program runs on a computer.

A further embodiment of the present disclosure is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present disclosure is an apparatus as described herein comprising a processor and the storage medium.

A further embodiment of the disclosure is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the disclosure comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

Embodiments may be based on using a machine-learning model or machine-learning algorithm. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g. sensor data, meta data and/or image data) may be preprocessed to obtain a feature vector, which is used as input to the machine-learning model.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g. a classification algorithm, a regression algorithm or a similarity learning algorithm. Classification algorithms may be used when the outputs are restricted to a limited set of values (categorical variables), i.e. the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are. Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g. a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g. based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g. of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input.

Alternatively, the machine-learning model may be a support vector machine, a random forest model or a gradient boosting model. Support vector machines (i.e. support vector networks) are supervised learning models with associated learning algorithms that may be used to analyze data (e.g. in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

### LIST OF REFERENCE SIGNS

100 OCT imaging system
102 light source
104 a beam splitter
106 reference arm
108, 114, 116 lenses
109, 115, 117 light beams
110 reference mirror
112 sample arm
118 diffraction grating
120 detector
122 intensity scan data
130 control system
132 processing means
134 image data
140 display means
142 OCT image
144 orientation of OCT scan
150 video imaging means
152 video image
154 orientation of subject
160 fiber optic cable
162 electrical cable
190 subject
192 tissue of interest
356 overlay
394 undesired object
400, 410, 420, 430, 440, 450 method steps
445 multitude of OCT scans

What is claimed is:

1. A control system for an imaging system for real-time imaging of a subject, using optical coherence tomography and video imaging, the control system being configured to:
control the imaging system to determine, from a video image of the subject, a position and/or an orientation of a tissue of interest in the subject,
control the imaging system to perform a scan of the subject by means of optical coherence tomography, wherein a position and/or an orientation of the scan is determined based on the position and/or orientation of the tissue of interest in the video image, and
provide an optical coherence tomography image of the subject, including the tissue of interest, based on the scan.

2. The control system of claim 1, further configured to control the imaging system to acquire the video image of the subject by means of video imaging means.

3. The control system of claim 1, further configured to determine, from the video image, the position and/or orientation of the tissue of interest in the subject by means of feature tracking and/or artificial intelligence.

4. The control system of claim 1, further configured to control the imaging system to perform a multitude of different scans of the subject, to identify undesired objects present in the subject, and to indicate the undesired objects within the video image of the subject.

5. The control system of claim 1, further configured to identify the undesired objects by means of feature tracking and/or artificial intelligence.

6. The control system of claim 1, wherein the subject includes or is an eye, and wherein the tissue of interest includes or is a Descemet's membrane or a graft Descemet's membrane.

7. The control system of claim 1, further configured to control the imaging system to scan the subject by performing a B-scan.

8. An imaging system for real-time imaging of a subject, comprising the control system of claim 1, optical coherence tomography means and video imaging means.

9. The imaging system of claim 8, configured for use during a surgical procedure being performed on the subject.

10. A method for imaging a subject in real-time, using optical coherence tomography and video imaging, comprising the following steps:
determining, from a video image of the subject, a position and/or an orientation of a tissue of interest in the subject,
performing a scan the subject by means of optical coherence tomography, wherein a position and/or an orientation of the scan is determined based on the position and/or orientation of the tissue of interest in the video image, and
providing an optical coherence tomography image of the subject, including the tissue of interest, based on the scan.

11. The method of claim 10, further comprising the following steps: performing a multitude of different scans of the subject, identifying undesired objects present in the subject, and indicating the undesired objects within the video image of the subject.

12. The method of claim 10, wherein the multitude of different scans of the subject is equally distributed with respect to an orientation and/or covers 360°.

13. The method of claim 10, wherein the subject includes or is an eye, and wherein the tissue of interest includes or is a Descemet's membrane or a graft Descemet's membrane.

14. The method of claim 10, wherein performing the scan of the subject comprises performing a B-scan.

15. A non-transient computer readable storage medium comprising a computer program including a program code for performing the method of claim 10, when the computer program is run on a processor or on the control system of claim 1.
